(19) United States
(12) Patent Application Publication
(10) Pub. No.: US 2008/0118911 A1
(43) Pub. Date: May 22, 2008

(54) Test Device for the In-Vitro Diagnosis of Multi-Analyte Tests and Use Thereof

(76) Inventors: Gerhard Hermann, Wurzburg (DE); Ilona Kuhlmann-Rabens, Creglingen (DE); Sabine Reder, Tauberbischofsheim (DE); Uwe Schobel, Wetzlar (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/661,216

(22) PCT Filed: Aug. 26, 2005
(86) PCT No.: PCT/EP05/09245

§ 371 Date: Sep. 25, 2007

(30) Foreign Application Priority Data
Aug. 27, 2004 (DE) 10 2004 041 659.1

(51) Int. Cl. C12Q 1/70; C12M 1/00; C40B 60/12; G01N 33/53; G01N 33/00; B32B 37/02; G01N 33/569; C12Q 1/68; C12Q 1/00

(52) U.S. Cl. 435/5; 435/287.1; 435/287.2; 506/39; 435/4; 435/6; 435/7.1; 435/7.32; 435/7.31; 435/7.92; 435/7.94; 435/7.93; 156/182; 156/60

(57) ABSTRACT

The present invention generally relates to the diagnosis of diseases and in particular novel methods and devices for an improved use of markers that are associated with diseases to be diagnosed. The present invention can find use in particular in the fields of diagnostics, diagnostic tests, and rapid tests.

### CROSS-REFERENCE TO RELATED APPLICATION

This application is a National Stage Application of International Application Number PCT/EP2005/009245, filed Aug. 26, 25005; which claims priority to German Application No. 10 2004 041 659.1, filed Aug. 27, 2004.

The present invention generally relates to the diagnosis of diseases and in particular to novel methods and devices for an improved use of markers that are associated with diseases to be diagnosed. The present invention can find specific use in the fields of diagnostics, diagnostic tests, and rapid tests.

### DESCRIPTION

A clinician needs exact and simple methods in order to provide a diagnosis for diseased or potentially diseased patients. Such methods enable the respective personnel to apply and monitor a more or less aggressive, and, in particular, more suitable medical treatment, especially for critically diseased patients.

Despite the fact that early tests were relatively simple, e.g. measuring the temperature of a patient with a thermometer, recent progresses in the research and technology have considerably broadened the progress and the number of diagnostic tests that are available. Currently, using microscopy in order to visually inspect a blood sample, laboratory technicians can, for example, determine both the amounts and types of white blood cells that are present in a patient, or automatic systems. The number of white blood cells and the differentiation of white blood cells per unit of volume can be utilized for the recognition of a microbial infection in a patient. Samples of body fluids, e.g., sputum, urine, blood, and wound samples can be cultivated on suitable plates, e.g., agar plates, so that bacteria, if present in the sample, can be detected and identified. In addition, using tests, such as, for example, the “Versant HCV RNA Qualitative Assay” (Bayer Diagnostics, Tarrytown, N.Y., USA), certain viral infections (such as, for example, hepatitis C (HCV)) can be identified. These tests and methods aid the clinician or medical doctor in the correct diagnoses of diseases, which can ensure a more suitable treatment.

Nevertheless, many common diagnostic tests and methods are unspecific, slow or imprecise. For example, tests that merely measure C-reactive protein are often used as indicator for pneumonia and other diseases. Nevertheless, such unspecific tests are not useable in critical situations, where the immediate treatment is required (Clyne et al. (1999) J. Emerg. Med. 17 (6): 1019-1025). In addition, tests that measure the sedimentation rate of erythrocytes can indicate changes in the protein content of blood and blood cells, nevertheless, the underlying agent, e.g., infection, arthritis, etc., can not be determined without further testing. Thus, such unspecific tests and methods are not able to support the clinician in an unambiguous determination with respect to the presence of, for example, an infection.

In particular in severely diseased patients, an unspecific, delayed or imprecise diagnosis can result in a delayed and/or non-suitable treatment, which can lead to further complications or even death. The non-suitable administration of antibiotics, for example, can lead to the development of antibiotic-resistant strains of bacteria. It was furthermore found for a delayed diagnosis that it increases the overall costs for the treatment of (infected) patients (Barenfanger et al. (2000) J. Clin. Microbiol. 38 (8): 2824-2828).

Usually, a patient would gain the largest benefit from a fast and exact diagnosis during the early phase of the disease. Such an improved diagnostics would allow for a suitable treatment in order to remove the pathogen, to reduce the symptoms, and/or to avoid/reduce further complications. Thus, tests that are suitable to measure a large number of markers or signals that relate to the immune response should be specific for a particular diagnosis or indication or, e.g., for a specific infectious pathogen, and should allow for an early diagnosis.

EP 0 725 081 describes the use of human Mx protein MxA monoclonal antibodies in the diagnosis of viral infections. It was, however, found that a diagnosis based on a single indicator of infection is not sufficient, and that two or more indicators are required in order to allow for an exact diagnosis of, e.g., an infection.

WO 02/103059 describes a method for determining the type of an infectious pathogen in a patient, who is suspected to suffer from an infectious pathogen. The method first includes a measuring of the amounts of a large number of markers in a sample of a body fluid of the patient. The markers of interest are generated by the patient as part of the innate immune response of the patient in response to the presence of the infectious pathogen, and are indicative for the type of the infectious pathogen in the patient. Then, a marker profile is identified, based on the measured amounts of said large number of markers. Finally, if the marker profile indicates an infection, then the type of infectious pathogen inside the patient is determined using said marker profile. In preferred embodiments, each single marker is either an mRNA or a protein. Methods for the identification of suitable markers and kits are also provided.

WO 02/090964 describes a method for measuring the performance of protein-microarrays. A multiplex micro-ELISA system is provided. WO 99/21012 describes colored latex particles for immunoassays.

US 2003-008410 describes immunoassay-methods and -devices that use flow-cytometry, coated latex-microspheres, and fluorochrome-labelled antibodies, in order to simultaneously analyse the presence and amounts of several antigens or antibodies in a sample. The use of particles (beads) as solid carriers for antigen-antibody reactions in order to detect antigens or antibodies in serum and other body fluids, is described as particularly attractive, when combined with flow-cytometry. It is intended to use beads in different sizes, colors or shapes, wherein each bead is provided with a different protein or antibody.

Multianalyte-technologies offer the possibility to determine several analytes as a panel in a combined test preparation. Thus, clinical diagnostics usually seeks for relevant markers in accordance with a particular specific problem, and said markers can be arranged, and placed into commerce as so-called “multiplex-technology”.

Both chip technology and bead-based multiplex-technique belong to the multianalyte-technologies, and also line blots can be found among these. Until now, only panels (collections of specific markers to be detected) are on the market that detect analytes of “one single kind”, i.e. antibodies against different infectious pathogens or subunits of a single pathogen, autoantibodies or antibodies against allergens or antigens, such as cytokines, hormones and/or certain tumor markers.

Known panels are, amongst others, the line-blots of the company Euroimmun (Germany); Euroline® autoimmune diagnostics-test strips including up to 14 antigens; Euroline® allergy diagnostics-test strips having different profiles, and test kits Euroline® infection serology, such as, for example, the so-called TORCH-profile. Furthermore, biochips of the company Euroimmun exist; such as, for example, the “biochip—mosaic infection serology”: EBV, and bead-based multiplex-systems.

Previous teachings, such as the ones of EP 0 725 081, US 2003-008410 or WO 02/103059 fail to describe diagnostic methods or tests that are based on a multitude of signals or markers that are suitably assembled, and can be quickly and effectively evaluated, in order to thus allow for a fast, cost effective, and exact diagnosis. Previous panels are not combined with supplementary parameters that, for example, belong to the so-called “unspecific” markers, whose determination can, nevertheless, surprisingly increase the significance of a test result—also in view of plausibility.

Until now, the combination of parameters from different fields of laboratory medicine into one test panel was not known (e.g. the fields of serology/immunology and clinical chemistry). The development of such tests thus represent an important progress in the field of diagnostic tests and medicine. The present invention fulfils these and further needs of the state of the art.

It is therefore a first object of the invention, to meet the above described needs in the state of the art by providing an improved test device for a faster and more exact diagnostics of multianalyte-tests. The device should furthermore be composed in such a way that it is suitable for a precise automated method which can be readily performed.

According to a first aspect, this object is solved by a test device for the in vitro diagnostics of analytes of interest in a sample, comprising a carrier matrix, to which a test panel of at least one specific marker, which is examined for a common diagnosis selected from the group of specific markers for inflammatory diseases, vaccination status, pregnancy, infectious diseases, allergies, viral diseases, blood bank-examinations, liquor-diagnostics, autoimmune-diseases, carcinoma, acute cardiac infarction, diabetes, alcoholism, and thyroid function and combinations thereof, is suitably attached, and to which furthermore at least one diagnostically unspecific marker is attached which is selected from the group of inflammatory markers, acute infection markers, chronic infection markers, and combinations thereof.

Until now, the combination according to the invention of parameters from different fields of the laboratory medicine and/or -diagnostics into a single test kit was not known. One example for such fields are serology/immunology and clinical chemistry. The test according to the invention includes supplementary parameters which essentially increase the significance of said test.

Using the multianalyte-technology according to the invention, for the first time the complete scope of immunological tests from, e.g., the fields of infection serology, tumor diagnostics, allergy, autoimmune diseases and clinical chemistry, following the same principle, can be effectively processed through the combination according to the invention, specifically in an automate. This universally applicable and unified methodology allows a very cost effective and fast performance of the scope of examinations in a test setting that is individually required for each patient. The resulting clear diagnosis allows for an early onset of therapeutic measures, or even prophylactic interventions. Thereby, severe, progressed or chronic diseases can be ameliorated.

Preferred is a test device for the in vitro diagnostics of the present invention, wherein the marker is selected from IgG, IgA, IgM or IgE or combinations thereof, wherein these markers are directed against certain antigens, or the marker itself is a particular antigen to be detected. Furthermore, markers can be nucleic acids, in particular sections of nucleic acids, such as, for example, RNA- or DNA-fragments of cells or viruses, such as, for example, in the case of CMV. Further preferably, the test panel (but also marker combinations) comprises antibodies or antigens that are present immobilized covalently or non-covalently. Techniques for an immobilization of the markers according to the invention onto surfaces depend from the type of surface used and the marker, and are very well known to the person of skill.

Further preferred is a test device for the in vitro diagnostics of the present invention, wherein the matrix is present in form of a test strip, a membrane, a biochip, latex particles and/or other particles. Particularly preferred is a test device according to the invention for the in vitro diagnostics, wherein the membrane is made of nylon, or the matrix is present in form of latex particles. The matrix can also consist of a suitable usual porous natural or synthetic (polymeric) material.

In a particular embodiment, the multianalyte-technology according to the invention is based on a principle that is well known from flow-cytometry. A big advantage of this technology comes from the fact that in most cases a respective instrumentation is already present at the site of the users. In addition, the diagnostic capacity of the immunological tests is increased through the increased sensitivity of the fluorescence-signaling, compared to the ELISA-color reaction. In contrast to the ELISA-technique, where the walls of the cavities of the microtiter plate are used for adsorptive coupling of the analyte or antibody, the multianalyte-technology according to the invention uses latex particles with a diameter of, e.g., 4.0 μm as a solid phase. The in principal distinguishable particles can be dyed with distinct intensities of a red fluorophore (see FIG. 1), whereby an assortment of, e.g., ten differently dyed latex particle-sets is generated.

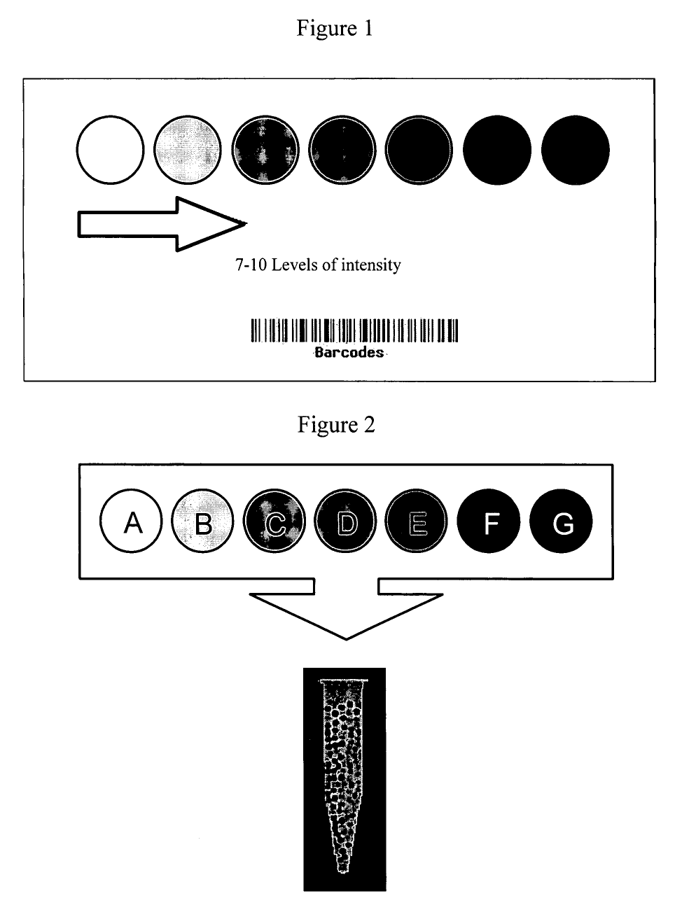

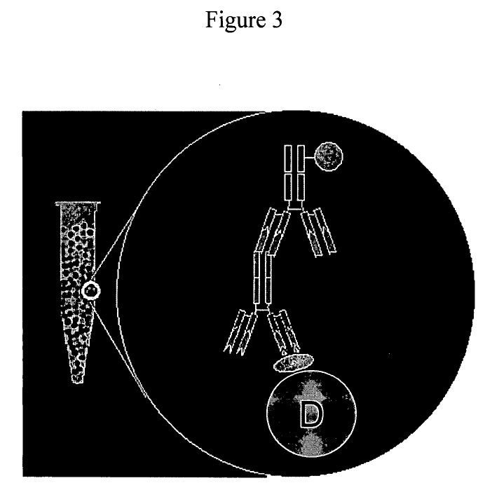

Each set is covalently coated with different antigens. In order to be able to perform several simultaneous examinations in one test setting, the antigen coated particle-sets are mixed into a cocktail. This mixture constitutes a multianalyte-plex according to the invention (see FIG. 2). During the incubation of the multianalyte-plex according to the invention with a patient serum, antibodies that are present bind to the latex particles—corresponding to their specificity. In the following incubation step, the conjugates that are labeled with a fluorescent dye react with the bound antibodies (see FIG. 3). The amount of bound conjugate molecules is proportional to the concentration of antibodies. Each set of latex particles of the multianalyte-plex according to the invention now carries a variable amount of the conjugate-fluorescence on its surface, and is analyzed by a flow-cytometer that is specialized for measurements of particles, a so-called particle-fluorescence-flow-instrument. These instruments analyse individual latex particles corresponding to their fluorescence, and, at the same time, can distinguish between three different fluorescent colors (orange, red, dark red). Using software, the device classifies the fluorescence of each particle into the matching particle-sets, and assigns the corresponding analyte determinations. The mean conjugate-fluorescence of each particle-set is a measure for the concentration of the respective analyte in the serum sample.

Thus, preferred is a test device for the in vitro diagnostics of the present invention, wherein the carrier matrix is composed of differently labeled carrier materials. It is further preferred that the carrier materials are colored in different intensities with a dye.

A particular aspect relates to a test device for the in vitro diagnostics of the present invention, wherein each carrier material is provided with at least one covalently or non-covalently immobilized antibody or antigen. Ideally, the test device for the in vitro diagnostics according to the invention comprises a defined mixture of suitably labeled carrier materials. Here, the use of latex particles in liquids allows for a fully flexible design of the tests into multiplex-test panels: Depending from the object for the individual patient, coated particle-sets can be combined into individual multianalyte-test-cocktails according to the invention. The diagnostic capacity of the multianalyte-tests is also improved through the higher sensitivity of the fluorescence-signaling (factor 5), compared to the ELISA-color reaction.

An essential advantage of the test device for the in vitro diagnostics of the present invention is achieved by the fact that different common test panel/markers are supplemented with specific additional parameters that a) are traditionally also determined in separate series of examinations during the respective diagnostic objectives, and/or b) that are regarded as indication-unspecific markers. Non-limiting examples for these are, e.g., the total increase of IgE as unspecific marker in the diagnosis of particular allergies using specific allergy markers (e.g. IgE against cat epithelium), or procalcitonin as general inflammatory marker in case of the diagnosis of particular infections. This additional parallel (simultaneous) measurement in accordance with examinations according to the invention improves the significance of results of specific tests. This combination according to the invention at the same time leads to several advantages, such as, amongst others, to an
Increase of the significance and plausibility of test results; e.g. the improvement of the significance regarding the stadium of the infection;
By using the test combinations, the result of the diagnosis can be accelerated in cases of acute clinical symptomatics; and
A cost reduction through the combination of parameters that otherwise are performed—in part in different departments—using different test systems.

According to the invention, amongst others, the following common specific marker-test combinations can be used with a test device, which then can be suitably supplemented.

Markers for the vaccination status can be selected from IgG against Tetanus toxoid, Diphtheria toxoid, Bordetella pertussis toxoid and Bordetella pertussis FHA. Markers for ToRCH can be selected from IgG or IgM against toxoplasmosis, Rubella, Cytomegalovirus, HSV1, HSV2, VZV and Parvovirus B19. Markers for the respiratory viral diagnosis can be selected from IgG or IgA against adenovirus, influenza A, influenza B, parainfluenza 1-3, RSV, and enteroviruses. Markers for the respiratory viral diagnosis can be selected from IgG or IgM against Chlamydia, Coxiella burnetii phase II, Legionella pneumophila, and Mycoplasma.

Markers for childhood diseases can be selected from IgG or IgM against Bordetella pertussis, measles, mumps, Rubella, and varizella-zoster virus (VZV).

Markers for zoonoses can be selected from IgG or IgM against Borellia p41 intern, OSP-C Borrelia burgdorferii, OSP-C Borrelia afzelii, OSP-C Borrelia garinii, p100, p41, and FSME.

Markers for Polio can be selected from IgG against polio 1, polio 2, and polio 3.

Markers for EBV can be selected from IgG or IgM against VCA p18, p23, EBNA, and early antigen.

Markers for Chlamydia can be selected from IgG or IgM against Chlamydia trachomatis, Chlamydia pneumoniae, and Chlamydia psittaci.

Markers for the gastrointestinal tract can be selected from IgG or IgA against Helicobacter pylorii, Campylobacter jejunii, and Yersinia enterocolitica.

Markers for fungal infections can be selected from IgG or IgM against Aspergillus fumigatus, and Candida albicans.

Markers for the diagnosis of rheumatism can be selected from rheumatic factors, HLA-markers, as well as autoimmune antibodies. Markers for autoimmune diseases (Sjögren-syndrome (SS-A), neonatal lupus erythematodes (LE), Sjögren-syndrome (SS-B)) can be selected from antibodies against extractable nuclear antigens, snRNP (ribonucleoprotein, systemic LE), ss-DNA, ds-DNA and c-ANCA (cytoplasmic-anti-neutrophil cytoplasm antibody). Markers for animal allergens can be selected from IgE against cat epithelia (e1), dog epithelia (e2), mite D. pteronyssinus (d1), mite D. farinae (d2) and the yeast Alternaria tenuis (m6). Marker for pollen allergens can be selected from IgE against true ragweed (w1), ribwort (w9), Bermuda grass (g2), blue grass (g8), oak tree (t7), elm tree (t8), and birch tree (t3).

Markers for the mamma carcinoma can be selected from CA15-3, CA15 549, and MCA.

Markers for the bronchial carcinoma can be selected from NSE, and CYFRA 21-1.

Markers for the acute cardiac infarction can be selected from myoglobin, troponin T, and creatin kinase.

Markers for the thyroid function can be selected from TSH, thyroxin (T4), triiodothyronin, free hormone fT3, free hormone fT4, and antibodies against thyroid peroxidase, thyreoglobulin, and TSH.

Markers for the pregnancy diagnostics can be selected from hcG, AFP, antibodies against Rubella, CMV, Toxoplasma gondii, HSV, parvovirus B19, and variella-zoster-virus

Markers for the liquor diagnostics can be selected from IgG or IgM against measles, Rubella, VZV, HSV FSME, EBV, enteroviruses, and Borrelia.

Markers for alcoholism can be selected from CDT (carbohydrate-deficient transferrin), gamma-GT, pancreatic elastase.

As mentioned above, these markers are supplemented with particular additional parameters that a) are traditionally co-determined in the respective diagnostic settings in separate series of examinations, and/or b) that are regarded as indication-unspecific markers. In the context of the present invention, these markers are understood as _{”}unspecific markers“. Non-limiting examples for this are, for example, the overall increase of IgE as unspecific marker in the diagnostics of particular allergies using specific allergy markers (e.g. IgE against cat epithelia), or procalcitonin as general inflammatory markers in case of the diagnostics of certain infections.

Thus, preferred is a test device for the in vitro diagnostics according to the invention, wherein this diagnostically _{”}unspecific marker“ is selected from albumin concentration, overall-immunoglobulin concentration, for example total-IgE-concentration in case of allergies, neopterin, C-reactive protein (CRP), cyclic citrullinated peptide, and procalcitonin, and combinations thereof.

Particularly preferred is the supplementation of the panel for liquor diagnostics, wherein the parameter that are obligatory to be determined are albumin- and overall-immunoglobulin concentration, together with neopterin for the distinction of acute or chronic infections of expired, healed up infections (example neuroborreliosis).

In infections caused by viruses, protozoa, and intracellular bacteria, the levels of neopterin increase in early stages of infection, even before the sero-conversion. This allows for the differential diagnosis of acute viral and bacterial infections, and thus for a reduction of the risk for infection in, e.g., blood transfusions.

Particularly preferred is furthermore the supplementation of the panel for a differential diagnosis of acute infections as well as a “blood bank-panel” for an exclusion of acute infections with “unspecific” parameters of infection or inflammation, such as, for example, C-reactive protein (CRP), a routine inflammatory parameter; in parallel to the erythrocyte sedimentation rate and protein electrophoresis, this serves for an estimation of the extent of the immune reaction.

Even more particularly preferred is the supplementation of the panel with markers for rheumatic factors, such as, for example, HLA-markers, together with the cyclic citrullinated peptide.

Furthermore preferred is the supplementation of the panel with procalcitonin, a relatively new prognostic parameter. It is selective for bacterial infections, and is essentially more sensitive than previous routine-inflammatory parameters. A suspicion for a bacterial infection is thus confirmed (differentiation between infections due to bacteria and viruses, differentiation of fever due to bacterial infection versus other causes). Procalcitonin is also indicative for systemic fungal infections, allowing for a differentiation of local fungal infections, and a use as prognostic marker in severe fungal infections.

A further aspect of the present invention relates to a method for producing a test device for the in vitro diagnostics according to the invention. This first comprises the attachment of a test panel of at least one specific marker that is examined for a common diagnosis selected from the group of specific markers for inflammatory diseases, vaccination status, pregnancy, infectious diseases, allergies, viral diseases, blood bank-examinations, liquor-diagnostics, autoimmune diseases, carcinoma, acute cardiac infarction, diabetes, alcoholism, and thyroid function, and combinations thereof onto a carrier matrix, and then the attachment of at least one diagnostically unspecific marker selected from the group of inflammatory markers, acute infection markers, chronic infection markers, and combinations thereof onto the same or a separate carrier matrix, and, optionally, combining of the test matrices from the first and the second step. The markers can be present either on separate test matrices or in discrete positions of a joint matrix.

Preferred is a method for producing a test device for the in vitro diagnostics according to the invention, wherein the marker for the common diagnosis is selected from IgG, IgA, IgM or IgE or combinations thereof, wherein these markers are directed against particular antigens or the marker itself is a particular antigen to be detected. Furthermore, the markers can be nucleic acids, in particular nucleic acid fragments, e.g. RNA- or DNA-fragments of cells or viruses, such as, for example, in case of CMV. Further preferred is a method for producing a test device for the in vitro diagnostics according to the invention, wherein the test panel/marker combination comprises antibodies or antigens that are immobilized covalently or non-covalently. The person of skill is aware of suitable techniques for immobilization (regarding this, see also above).

Further preferred is a method for producing a test device for the in vitro diagnostics according to the invention, wherein the matrix is present in form of a test strip, a membrane, a biochip, latex particles and/or other particles. Particularly preferred is a method for producing a test device for the in vitro diagnostics according to the invention, wherein the membrane is made of nylon or the matrix is present in form of latex particles. The matrix can also consist of a suitable common porous natural or synthetic (polymeric) material. Nevertheless, membranes according to the invention can also be made of other materials (e.g. nitrocellulose).

Further preferred is a method for producing a test device for the in vitro diagnostics according to the invention, wherein the carrier matrix is composed of differently labeled carrier materials. In addition to dyes, also other labels (or markers) can be used that are detectable and known in the field of immunodiagnostics. Even further preferred is a method for producing a test device for the in vitro diagnostics according to the invention, wherein the carrier materials are colored with a dye in different intensities. This, for example, allows for the generation of a distinguishable (“bar coded”) set of latex particles through dyeing of the particle with a dye in different intensities. In addition, different, e.g. three, fluorescent colors (orange, red, dark red) can be used. Using software, a respective device (see above) classifies the fluorescence of each particle into the matching sets of particles, and assigns the corresponding analyte-determinations.

A particular aspect relates to a method for producing a test device for the in vitro diagnostics according to the invention, wherein each carrier material is provided with at least one antibody or antigen, immobilized covalently or non-covalently. Reasonably, the method according to the invention comprises a defined mixture of suitably labeled carrier materials. The use of latex particles in fluids allows for a completely flexible design of the tests into multiplex-test panels: in accordance with the respective object, for each patient coated particle-sets can be arranged into individual multianalyte-test-cocktails according to the invention.

An even further aspect of the present invention then relates to a method for the in vitro diagnostics of analytes of interest in a sample, comprising providing of a test device for the in vitro diagnostics according to the present invention, contacting of a sample to be analyzed with the markers on the test device, and detecting a reaction of the sample with the markers on the test device in a suitable manner.

Preferred is a method for the in vitro diagnostics according to the invention, wherein the marker is selected from IgG, IgA, IgM or IgE or combinations thereof, wherein these markers are directed against certain antigens, or the marker itself is a particular antigen to be detected. Furthermore, markers can be nucleic acid, in particular nucleic acid fragments, e.g., RNA- or DNA-fragments of cells or viruses, such as, for example, in case of CMV. Markers for the common diagnosis can be all specific antibodies or antigens, such as, e.g., tumor markers that are detected as specific analytes. Further preferred is a method for in vitro diagnostics according to the invention, wherein the test panel comprises antibodies or antigens that are immobilized covalently or non-covalently. The person of skill is aware of suitable techniques for immobilization (regarding this, see also above).

Further preferred is a method for the in vitro diagnostics according to the invention, wherein marker combinations, as given above, are detected as markers. The sample that is used for the method for the in vitro diagnostics according to the invention is preferably selected from liquor, urine, semen, swaps, biopsies, sputum, breast-nipple aspirate, sweat, serum, and blood and parts thereof, and mixtures thereof.

Particularly preferred is a method for the in vitro diagnostics according to the invention, wherein detecting a reaction of the sample with the markers comprises the formation of a visually observable color reaction. The person of skill is well aware of respective color reactions. Further preferred is a method for the in vitro diagnostics according to the invention, wherein detecting a reaction of the sample with the markers comprises an ELISA. According to the invention, the detection of a reaction of the sample with the markers can comprise a sandwich assay or competitive assay. The respective composition of such tests is known to the person of skill (regarding this, see also FIG. 3).

Further preferred is a method for the in vitro diagnostics according to the invention, wherein the carrier matrix is composed of differently labeled carrier materials. In parallel to dyes, also other detectable groups can be used that are known in the field of immunodiagnostics. Even further preferred is a method for the in vitro diagnostics according to the invention, wherein the carrier materials are colored with a dye in different intensities. This e.g. allows for the generation of a distinguishable (“bar coded”) set of latex particles through dyeing of the particles with a dye in different intensities. In addition, different, e.g. three, fluorescent colors (orange, red, dark red) can be used. Using a a software, a special device (see above) classifies the fluorescence of each particle into the matching sets of particles and assigns the corresponding analyte-determinations.

A particular aspect relates to a method for the in vitro diagnostics of the present invention, wherein each carrier material is provided with at least one antibody or antigen, immobilized covalently or non-covalently. Reasonably, the method according to the invention comprises a defined mixture of suitably labeled carrier materials.

The use of latex particles in liquids advantageously allows for a completely flexible design of the tests into multiplex-test panels: coated sets of particles can be composed into individual multianalyte test-cocktails according to the invention in accordance with the problem-setting for the specific patient.

In a particular embodiment, the multianalyte-technology according to the invention is based on a principle known from flow cytometry. A big advantage of this technology has to be seen in the fact that in most cases respective devices are already available for the users. In addition, the diagnostic capability of the immunological tests is increased through the increased sensitivity of the fluorescence-signaling compared to the ELISA-color reaction.

Thus, preferred is a method for the in vitro diagnostics of the invention, comprising the detection of the labeled carrier materials by means of particle-fluorescence-flow cytometry. In contrast to the ELISA-technique, where the walls of the cavities of the microtiter plate are used for the adsorptive coupling of the analytes or antibodies, multianalyte-technology according to the invention uses latex particles with a diameter of 4.0 um as solid phase. The principally non-distinguishable particles are dyed with distinct intensities of a red fluorophore (see FIG. 1), whereby an assortment of ten differently colored sets of latex particles is generated.

Each set is covalently or adsorptively coated with different antigens, antibodies or nucleic acids. In order to now be able to perform several examinations in parallel in one test setting, the coated sets of particles are admixed into a cocktail. This mixture represents the multianalyte-plex according to the invention (see FIG. 2). During the incubation of the multianalyte-plex according to the invention with a patient serum or other patient material, analytes as present bind to latex particles—in accordance with their specificity. In the following incubation step, the conjugate that are labeled with a fluorescent dye react with the bound analyte (see FIG. 3). The amount of bound conjugate molecules is proportional to the analyte-concentration. The multianalyte plex according to the invention now carries a variable amount of the conjugate-fluorescence on its surface for each set of latex particles, and is analyzed by a flow cytometer that is specialized for particle-measurements, a so-called particle-fluorescence-flow-measuring apparatus. These measuring apparatuses analyse individual latex particles in accordance with their fluorescence, and can simultaneously distinguish between three different fluorescent colors (orange, red, dark red). With the aid of a software the device classifies the fluorescence of each particle into the matching set of particles, and assigns the corresponding analyte determinations. The mean conjugate-fluorescence of each set of particles is a measure for the concentration of the respective analyte in the serum sample.

Advantages of the multianalyte-technology according to the invention, amongst others, lie in the fact that the multianalyte technology according to the invention can deliver important medicinal-diagnostic information for the whole field of immunology, e.g. for the serology of infections and for the detection of autoimmune diseases, allergies, tumor markers and hormones. For this, the sera of different collectives of patients are examined. Through multiplexing, new patterns of the sero-reactivity are generated that markedly improve the predictive information of diagnostic data. In some cases, the diagnostic significance can also be expressed as quotient of two or more sero-reactivities, here, the multianalyte-test-run shows a markedly improved precision. The use of latex particles in liquids allows for a completely flexible design of the tests into multiplex-test panels: in accordance with the problem-setting for the respective patient, coated sets of particles can be composed into individual multianalyte test-cocktails according to the invention.

In this description and in the following claims, the following terminology in agreement with the below indicated definitions is used.

A “marker” or _{”}analyte“ in the sense of the present invention are analytes that are specific for certain physiological or pathological conditions are, e.g. antibodies. Markers can be selected from IgG, IgA, IgM or IgE or combinations thereof that are directed against specific antigens, or themselves can be particular antigens to be detected. Furthermore, markers can be nucleic acids, in particular nucleic acid fragments, e.g. RNA- or DNA-fragments of cells or viruses, such as, for example, CMV.

An “unspecific marker” in the sense of the present invention is a marker that a) is traditionally co-determined in separate series of examinations during the corresponding diagnostic problem-setting, and/or b) is regarded as an indication-unspecific marker. Non-limiting examples for these are, e.g., the overall increase of IgE as unspecific marker in the diagnostics of certain allergies using specific allergy markers (e.g. IgE against cat epithelia), or procalcitonin as general inflammatory markers in case of the diagnostics of certain infections. Other unspecific markers are selected from the group of markers for inflammatory diseases, vaccination status, pregnancy, infectious diseases, allergies, viral diseases, blood bank-examination, liquor diagnostics, autoimmune diseases, carcinoma, acute cardiac infarction, diabetes, alcoholism, thyroid function, inflammatory markers, markers of acute infection, markers of chronic infection, and combinations thereof.

A “specific marker” in the sense of the present invention is a marker that is commonly used as specific analyte of a specific diagnostic indication (problem). Examples are mentioned above, and relate to, e.g., specific tumor antigens or immunoglobulins, such as, e.g., IgG against Tetanus toxoid, IgM against Borrelia p41 intern, IgM against VCA p18, and many others.

“Patient” as used herein, refers to an organism, preferably a mammal, further preferred a human. The present invention—in addition to other aspects—allows for a determination of the type of infectious pathogen being present in a patient, and/or the physiological or pathological condition of a patient in view of a targeted diagnostic or therapeutic problem-setting (e.g. disease).

As used herein, the terms “patient sample”, “a sample obtained from a patient” and “a sample obtained from an individual” are used interchangeably, and include any sample that was obtained from a patient or other individual. Thus, the sample can be a solid tissue sample, e.g., a biopsy sample, a liquid sample, e.g., a blood sample, or any other patient sample that is commonly medically used. In some embodiments of the invention, the sample is a sample of “body fluid”, a sample of lymphatic fluid, cell lysates, milk, plasma, salvia, semen, serum, spinal fluid, tears, whole blood, fractions of whole blood, wound samples, the external parts of the skin, and the secretions of the respiratory, intestinal, and genitourinary tracts. Preferably, the sample is blood, sputum, urine or fractions of whole blood.

The term “binding conditions” is intended to mean those conditions of time, temperature, and pH, and the required amounts and concentrations of reactants and reagents that are sufficient in order to allow for a binding between binding pairs, e.g., an antibody to its protein that has the corresponding epitope. As is well known in the state of the art gut, the conditions of time, temperature, and pH as required for a binding depend from the size of each member of the binding pair, the affinity between the binding pair, and the presence of other materials in the reaction mixture. The actual conditions that are required for each binding step, are well known in the state of the art or can be readily determined without undue burden.

Typical conditions for binding for the majority of biomolecules, e.g., antibodies to a protein, that has the corresponding epitope, include the use of solutions that are buffered to a pH of about 7 to about 8.5, and performed at temperatures of about 22° C. to about 60° C., and preferably of about 30° C. to about 55° C. for a period of time of about 1 second to about 1 day, preferably of about 10 minutes to about 16 hours, and most preferred of about 15 minutes to about 3 hours. “Conditions for binding” also require an effective buffer. Any buffer that is compatible, i.e., chemically inert in vie of the biomolecules and other components, and still allows for a binding between the binding pair, and essentially inhibits unspecific binding, can be used.

The term “protein” means a polymer, wherein the monomers are amino acids that are linked by amide bonds. The protein can be composed of at least about 5 amino acids, further common of at least about 10 amino acids, and most commonly of at least about 50 amino acids.

The term “coupled”, as used herein, relates to the attachment through covalent bonds or through non-covalent interactions (e.g., hydrophobic interactions, hydrogen bridges, etc.). Covalent bonds can be, for example, ester, ether, phosphoester, amide, peptide, imide, carbon-sulfur bonds, carbon-phosphor bonds, and the like. Methods for coupling of proteins to substrates and matrices are well known in the state of the art, and, for example, include a blotting of the proteins to the substrate.

The term “substrate” or “matrix” relates to any solid or semi-solid surface, to which a desired binding partner shall be anchored. Suitable substrate materials can be any material that can immobilize a biomolecule, e.g. a protein, and, e.g., include, glass (e.g. for object slides), nitrocellulose (e.g. in membranes), plastics, including polyvinylchloride (e.g. in sheets or microtiter wells), polystyrene latex (e.g. in beads or microtiter plates), polyvinylidine fluoride (e.g. in microtiter plates), and polystyrene (e.g. in beads), metal, polymeric gels, and the like.

The term “label”, as used herein, relates to any atom or moiety that can be used in order to generate a detectable (preferably quantifiable) signal, and onto which a biomolecule, e.g. a protein, can be attached.

The term “test panel” as used herein, relates to a arrangement of a group of parameters to be examined that are grouped and examined in view of certain targeted diagnostic problem-settings. Such problem-settings, in particular, relate to inflammatory diseases, vaccination status, pregnancy, infectious diseases, allergies, viral diseases, blood bank-examination, liquor diagnostics, autoimmune diseases, carcinoma, acute cardiac infarction, diabetes, alcoholism, thyroid function, inflammatory markers, markers of acute infection, markers of chronic infection, and combinations thereof. A test panel can be present physically on a matrix or on several matrices in the test according to the invention (i.e., for example, on different latex beads).

The invention shall now be further explained in the following based on examples with reference to the accompanying Figures, without being limited thereto. In the Figures,

FIG. 1: shows the generation of a preferred distinguishable (“bar coded”) set of latex particles through dyeing of the particles using one dye in different intensities,

FIG. 2: shows the generation of a preferred multianalyte-plex according to the invention by admixing the sets of latex particles that are coated with antigen, and

FIG. 3: shows the scheme of the process for a preferred multianalyte test system according to the invention. In a first reaction step, the latex particle bind the specific antibodies that are present in the serum (e.g. green-blue). In the second step, the conjugate antibodies that are labeled with a fluorescent dye (e.g. red-orange) are binding.

### EXAMPLE

In this example, the multianalyte-technology according to the invention is based on a principle that is known from flow cytometry. First, latex particles with a diameter of 4.0 um are used as solid phase. These in principle non-distinguishable particles are then dyed with different/distinct intensities of a red fluorophore (see FIG. 1), whereby an assortment of ten differently colored sets of latex particles is generated.

Each set is then covalently coated with different antigens. For several simultaneous examinations, the antigen-coated sets of particles are then admixed into a defined cocktail.

This mixture represents a multianalyte-plex (or also panel, see FIG. 2) according to the invention. Then, the incubation of the multianalyte-plex according to the invention with a patient serum takes place, whereby antibodies as present—in accordance with their specificity—bind to latex particles. In the following incubation step, the conjugates that are labeled with a fluorescent dye react with the bound antibodies (see FIG. 3). Thereby, the amount of bound conjugate molecule is proportional to the concentration of antibody.

For each set of latex particles, the multianalyte plex according to the invention now carries a variable amount of the conjugate-fluorescence on its surface, and is analyzed by a flow cytometer that is specialized for particle-measurements, a so-called particle-fluorescence-flow-measuring apparatus. These measuring apparatuses analyse individual latex particles in accordance with their fluorescence, and at the same time can distinguish three different fluorescent dyes (orange, red, dark red). Using a software, the device classifies the fluorescence of each particle into the matching sets of particles, and assigns the corresponding analyte determinations. The mean conjugate-fluorescence of each set of particles is a measure for the concentration of the corresponding analyte in the serum sample.

This technology is equivalently used in the overall field of immunology, e.g. for a serology of infections and for a detection of autoimmune diseases, allergies, tumor markers, and hormones. For this, the sera of different collectives of patients are examined. Through multiplexing, new patterns of seroreactivity are generated that markedly improve predictive information of diagnostic data. In some cases, the diagnostic significance is also expressed as a quotient of two or more seroreactivities, here, the multianalyte-test run shows a markedly improved precision compared to common methods, in particular the rate of false-positives can be markedly reduced due to the fact that these are recognized and eliminated.

1. A test device for the in vitro diagnostics of analytes of interest in a sample, comprising
a) a carrier matrix, to which,
b) a test panel of at least one diagnostically specific marker that is examined for a common diagnosis selected from the group of inflammatory diseases, vaccination status, pregnancy, infectious diseases, allergies, viral diseases, blood bank-examinations, liquor-diagnostics, autoimmune-diseases, carcinoma, acute cardiac infarction, diabetes, alcoholism, and thyroid function and combinations thereof, and
c) at least one diagnostically unspecific marker are suitably attached.

2. The test device for in vitro diagnostics according to claim 1, wherein at least one marker is selected from specific IgG, IgA, IgM or IgE, antibodies, parts of antibodies, recombinant antigen-binding peptides, nucleic acids or their fragments or combinations thereof.

3. The test device for in vitro diagnostics according to claim 1, wherein the test panel comprises antibodies or antigens that are immobilized covalently or non-covalently to the carrier matrix.

4. The test device for in vitro diagnostics according to claim 1, wherein the matrix is a test strip, a membrane, a biochip, latex particles, and/or other particles.

5. The test device for in vitro diagnostics according to claim 4, wherein the membrane is made of nylon or nitrocellulose.

6. The test device for in vitro diagnostics according to claim 1, wherein the matrix consists of a porous material.

7. The test device for in vitro diagnostics according to claim 1, wherein the carrier matrix is composed of differently labeled carrier materials.

8. The test device for in vitro diagnostics according to claim 7, wherein the carrier materials are colored with a dye in different intensities.

9. The test device for in vitro diagnostics according to claim 7 wherein each carrier material is provided with at least one antibody or antigen, immobilized covalently or non-covalently.

10. The test device for in vitro diagnostics according to claim 1, comprising a defined mixture of labeled carrier materials.

11. The test device for in vitro diagnostics according to claim 1, comprising at least one of
diagnostically specific marker for the vaccination status selected from IgG against Tetanus toxoid, Diphtheria toxoid, Bordetella pertussis toxoid, or Bordetella pertussis FHA,
a diagnostically specific marker for ToRCH selected from IgG or IgM against toxoplasmosis, Rubella, Cytomegalovirus, HSV1, HSV2, VZV, or parvovirus B19,
a diagnostically specific marker for respiratory viral diagnosis selected from IgG or IgA against adenovirus, influenza A, influenza B, parainfluenza 1-3, RSV, or enteroviruses,
a diagnostically specific marker for respiratory viral diagnosis selected from IgG or IgM against Chlamydia, Coxiella burnetii phase II, Legionella pneumophila, or Mycoplasma,
a diagnostically specific marker for childhood diseases selected from IgG or IgM against Bordetella pertussis, measles, mumps, Rubella and varizella-zoster virus (VZV),
a diagnostically specific marker for zoonoses selected from IgG or IgM against Borrelia p41 intern, OSP-C Borrelia burgdorferii, OSP-C Borrelia afzelii, OSP-C Borrelia garinii, p100, p41 or FSME,
a diagnostically specific marker for polio selected from IgG against polio 1, polio 2, or polio 3,
a diagnostically specific marker for EBV selected from IgG or IgM against VCA p18, p23, EBNA, or early antigen,
a diagnostically specific marker for Chlamydia selected from IgG or IgM against Chlamydia trachomatis, Chlamydia pneumoniae or Chlamydia psittaci, and
a diagnostically specific marker for the gastrointestinal tract selected from IgG or IgA against Helicobacter pylorii, Campylobacter jejunii or Yersinia enterocolitica.

12. The test device for in vitro diagnostics according to claim 1. comprising a diagnostically specific marker for fungal infections selected from IgG or IgM against Aspergillus fumigatus or Candida albicans.

13. The test device for in vitro diagnostics according to claim 1, comprising at least one of the following: a diagnostically specific marker for the rheumatism-diagnosis selected from rheumatism-factors, HLA-markers, as well as autoimmune antibodies,
a diagnostically specific marker for autoimmune diseases, Sjögren-syndrome (SS-A), neonatal lupus erythematodes (LE), Sjögren-syndrome (SS-B) selected from antibodies against extractable nuclear antigens, snRNP (ribonucleoprotein, systemic LE), ss-DNA, ds-DNA, and c-ANCA (cytoplasmatic-anti-neutrophil cytoplasm antibody),
a diagnostically specific marker for animal allergens selected from IgE against cat epithelia (e1), dog epithelia (e2), mite D. pteronyssinus (d1), mite D. farinae (d2) or yeast Alternaria tenuis (m6), and
a diagnostically specific marker for pollen allergens selected from IgE against true ambrosia (w1), ribwort (w9), Bermuda grass (g2), bluegrass (g8), oak tree (t7), elm tree (t8), and birch tree (t3).

14. The test device for in vitro diagnostics according to claim 1, comprising a diagnostically specific marker for the mamma carcinoma selected from CA 15-3, CA 15 549 and MCA, and
a diagnostically specific marker for the bronchial carcinoma is selected from NSE and CYFRA 21-1.

15. The test device for in vitro diagnostics according to claim 1, wherein the diagnostically specific marker for the acute cardiac infarction is selected from myoglobin, troponin T, and creatin kinase.

16. The test device for in vitro diagnostics according to claim 1, wherein the diagnostically specific marker for the thyroid function is selected from TSH, thyroxin (T4), triiodothyronin, free hormone fT3, free hormone fT4 and antibodies against thyreoperoxidase, thyreoglobulin or TSH.

17. The test device for in vitro diagnostics according to claim 1, wherein the diagnostically specific marker for the pregnancy diagnostics is selected from IgG, IgM or IgA against hcG, AFP, Rubella, CMV, Toxoplasma gondii, HSV, parvovirus B19 or varizella-zoster-virus.

18. The test device for in vitro diagnostics according to claim 1, wherein the diagnostically specific marker for the liquor diagnostics is selected from IgG or IgM against measles, Rubella, VZV, HSV, enteroviruses, EBV, FSME or Borrelia.

19. The test device for in vitro diagnostics according to claim 1, wherein the diagnostically specific marker for alcoholism is selected from CDT (carbohydrate-deficient transferrin), Gamma-GT, and pancreatic elastase alpha-amylase.

20. The test device for in vitro diagnostics according to claim 1 comprising a diagnostically specific marker is selected from the group of inflammatory markers, acute infection markers, chronic infection markers, and combinations thereof.

21. A method for producing a test device for in vitro diagnostics, comprising
a) attaching a test panel of at least two diagnostically specific markers that are examined for a common diagnosis selected from the group of inflammatory diseases, vaccination status, pregnancy, infectious diseases, allergies, viral diseases, blood bank-examinations, liquor-diagnostics, autoimmune-diseases, carcinoma, acute cardiac infarction, diabetes, alcoholism, and thyroid function and combinations thereof onto a carrier matrix,
b) attaching at least one diagnostically unspecific marker onto the same or a separate carrier matrix, and
c) optionally, combining of the test matrices from step a) and b).

22. The method for producing a test device for in vitro diagnostics according to claim 21, wherein at least one marker is selected from specific IgG, IgA, IgM or IgE, antibodies, parts of antibodies, recombinant antigen-binding peptides, nucleic acids or their fragments or combinations thereof.

23. The method for producing a test device for in vitro diagnostics according to claim 21, wherein the test panel comprises antibodies or antigens that are immobilized covalently or non-covalently to the carrier matrix.

24. The method for producing a test device for in vitro diagnostics according to claim 21, wherein the carrier matrix is a test strip, a membrane, a biochip, latex particles, and/or other particles.

25. The method for producing a test device for in vitro diagnostics according to claim 24, wherein the membrane consists of nylon or nitrocellulose.

26. The method for producing a test device for in vitro diagnostics according to claim 21, wherein the carrier matrix consists of a porous material.

27. The method for producing a test device for in vitro diagnostics according to claim 21, wherein the carrier matrix is composed of differently labeled carrier materials.

28. The method for producing a test device for in vitro diagnostics according to claim 27, wherein the carrier materials are colored with a dye in different intensities or are differently labeled with a radioactive, magnetic and/or luminescent marker.

29. The method for producing a test device for in vitro diagnostics according to claim 27, wherein each carrier material is provided with at least one antibody or antigen, immobilized covalently or non-covalently.

30. The method for producing a test device for in vitro diagnostics according to claim 21, wherein the labeled carrier materials are combined as a defined mixture.

31. A method for the in vitro diagnostics of analytes of interest in a sample, comprising
a) providing a test device for the in vitro diagnostics
wherein the test device comprises:
i) a carrier matrix, to which,
ii) a test panel of at least one diagnostically specific marker that is examined for a common diagnosis selected from the group of inflammatory diseases, vaccination status, pregnancy, infectious diseases, allergies, viral diseases, blood bank-examinations, liquor-diagnostics, autoimmune-diseases, carcinoma, acute cardiac infarction, diabetes, alcoholism, and thyroid function and combinations thereof, and
iii) at least one diagnostically unspecific marker
are suitably attached,
b) contacting a sample with the markers on the test device, and
c) detecting a reaction in the sample with the markers on the test device.

32. The method for in vitro diagnostics according to claim 31, wherein the marker is selected from specific IgG, IgA, IgM or IgE, antibodies, parts of antibodies, recombinant antigen-binding peptides, nucleic acids or their fragments or combinations thereof.

33. The method for in vitro diagnostics according to claim 31, wherein the test panel comprises antibodies or antigens that are present immobilized covalently or non-covalently.

34. The method for in vitro diagnostics according to claim 31, wherein the device comprises at least one of:
a diagnostically specific marker for the vaccination status selected from IgG against Tetanus toxoid, Diphtheria toxoid, Bordetella pertussis toxoid, or Bordetella pertussis FHA
a diagnostically specific marker for ToRCH selected from IgG or IgM against toxoplasmosis, Rubella, Cytomegalovirus, HSV1, HSV2, VZV, or parvovirus B19,
a diagnostically specific marker for respiratory viral diagnosis selected from IgG or IgA against adenovirus, influenza A, influenza B, parainfluenza 1-3, RSV, or enteroviruses,
a diagnostically specific marker for respiratory viral diagnosis selected from IgG or IgM against Chlamydia, Coxiella burnetii phase II, Legionella pneumophila, or Mycoplasma,
a diagnostically specific marker for childhood diseases selected from IgG or IgM against Bordetella pertussis, measles, mumps, Rubella and varizella-zoster virus (VZV),
a diagnostically specific marker for zoonoses selected from IgG or IgM against Borrelia p41 intern, OSP-C Borrelia burgdorferii, OSP-C Borrelia afzelii, OSP-C Borrelia garinii, p100, p41 or FSME,
a diagnostically specific marker for polio selected from IgG against polio 1, polio 2, or polio 3,
a diagnostically specific marker for EBV selected from IgG or IgM against VCA p18, p23, EBNA, or early antigen,
a diagnostically specific marker for Chlamydia selected from IgG or IgM against Chlamydia trachomatis, Chlamydia pneumoniae or Chlamydia psittaci,
a diagnostically specific marker for the gastrointestinal tract selected from IgG or IgA against Helicobacter pylorii, Campylobacter jejunii or Yersinia enterocolitica,
a diagnostically specific marker for fungal infections selected from IgG or IgM against Asperillus fumigatus or Candida albicans,
a diagnostically specific marker for the rheumatism-diagnosis selected from rheumatism-factors, HLA-markers, and autoimmune antibodies,
a diagnostically specific marker for autoimmune diseases, Sjögren-syndrome (SS-A), neonatal lupus erythematodes (LE), Sjögren-syndrome (SS-B) selected from antibodies against extractable nuclear antigens, snRNP (ribonucleoprotein, systemic LE), ss-DNA, ds-DNA, and c-ANCA (cytoplasmatic-anti-neutrophil cytoplasm antibody),
a diagnostically specific marker for animal allergens selected from IgE against cat epithelia (e1), dog epithelia (e2), mite D. pteronyssinus (d1), mite D. farinae (d2) or yeast Alternaria tenuis (m6),
a diagnostically specific marker for pollen allergens selected from IgE against true ambrosia (w1), ribwort (w9), Bermuda grass (g2), bluegrass (g8), oak tree (t7), elm tree (t8), and birch tree (t3),
a diagnostically specific marker for the mamma carcinoma selected from CA 15-3, CA 15 549 and MCA,
a diagnostically specific marker for the bronchial carcinoma is selected from NSE and CYFRA 21-1
a diagnostically specific marker for the acute cardiac infarction selected from myoglobin, troponin T, and creatin kinas,
a diagnostically specific marker for the liquor diagnostics selected from IgG or IgM against measles, Rubella, VZV, HSV, enteroviruses, EBV, FSME or Borrelia,
a diagnostically specific marker for alcoholism selected from CDT (carbohydrate-deficient transferring Gamma-GT, and pancreatic elastase alpha-amylase,
a diagnostically specific marker selected from the group of inflammatory markers, acute infection markers, chronic infection markers, and combinations thereof, and
a diagnostically specific marker for the pregnancy diagnostics selected from IgG, IgM or IgA against hcG, AFP, Rubella, CMV, Toxoplasma gondii, HSV, parvovirus B19 or varizella-zoster-virus.

35. The method for in vitro diagnostics according to claim 31, wherein the sample is selected from liquor, urine, semen, swabs, biopsies, sputum, breast-aspiration fluid, sweat, serum and blood and parts thereof, and mixtures thereof.

36. The method for in vitro diagnostics according to claim 31, wherein the detection of a reaction of the sample with the markers comprises the formation of a visually readable color reaction.

37. The method for in vitro diagnostics according to claim 31, wherein the detection of a reaction of the sample with the markers comprises an ELISA.

38. The method for in vitro diagnostics according to claim 31, wherein the detection of a reaction of the sample with the markers comprises a sandwich-assay or competitive assay.

39. The method for in vitro diagnostics according to claim 31, wherein a carrier matrix is detected that is composed of differently labeled carrier materials.

40. The method for in vitro diagnostics according to claim 39, wherein the carrier materials are colored with a dye in different intensities and/or different colors of fluorescence.

41. The method for in vitro diagnostics according to claim 31, wherein each carrier material is provided with at least one antibody or antigen, immobilized covalently or non-covalently.

42. The method for in vitro diagnostics according to claim 31, comprising the detection of the labeled carrier materials by means of particle fluorescence-flow cytometry.
